(19) United States
(12) Patent Application Publication
(10) Pub. No.: US 2021/0051836 A1
(43) Pub. Date: Feb. 25, 2021

(54) AUTONOMOUS UNMANNED GROUND VEHICLE FOR PEST CONTROL

(71) Applicant: ELEOS ROBOTICS INC., Surrey (CA)

(72) Inventors: Yahoel VAN ESSEN, White Rock (CA); Gordon HO, Richmond (CA)

(21) Appl. No.: 16/963,907

(22) PCT Filed: Jan. 24, 2019
(86) PCT No.: PCT/CA2019/050087

§ 371 Date: Jul. 22, 2020

Related U.S. Application Data
(60) Provisional application No. 62/622,016, filed on Jan. 25, 2018.

(51) Int. Cl. A01B 69/00; A01M 21/04; A01B 69/04; G05B 19/4155

(52) U.S. Cl. CPC A01B 69/001; A01M 21/04; G05B 2219/50393; G05B 19/4155; A01B 69/008

(57) ABSTRACT

Herein provided is an autonomous unmanned ground vehicle (AUGV) and handheld device for pest control. The AUGV comprises a chassis with a drive mechanism to displace the AUGV among a plurality of plants comprising at least one weed; an image-capture device to obtain images of the plurality of plants; a motorized arm with a free end displaceable with respect to the chassis; a microwave emitter mounted to the free end, displaceable therewith, and operable to emit microwaves; and a control system to operate at AUGV. The control system comprises a processing unit; and a memory having stored thereon instructions to cause the AUGV to perform: independently navigating the chassis among the plurality of plants; identifying the at least one weed in the images; displacing the motorized arm to position the free end in proximity to the at least one weed; and emitting microwaves toward the at least one weed.

### CROSS-REFERENCE TO RELATED APPLICATIONS

This application claims priority to U.S. provisional patent application having Ser. No. 62/622,016 and filed on Jan. 25, 2018, the entire contents of which are incorporated by reference herein.

### TECHNICAL FIELD

The present disclosure relates generally to autonomous vehicles, and more specifically to autonomous vehicles for controlling pests.

### BACKGROUND

Plant agriculture has always been plagued by various pests which affect the ability of farmers to maximize the production of crops. Common types of pests include weeds, cryptogams, and other undesirable plants, insects, rodents and other vermin, and bacteria. Pests can interfere with the ability of crops to grow and/or reproduce, spoil the harvestable portions of crops, infect crops with diseases, etc., costing farmers considerable amounts of revenue and reducing the total amount of crops available for consumption.

Pest control has traditionally taken two main forms. A first is physical removal of undesirable plants or vermin. Undesirable plants can be uprooted or cut, and vermin can be captured and eliminated, or prevented from access to the desirable plants by physical means, such as fences, walls, and the like. A second is chemical treatment, where a pesticide or other chemical substance is applied to crops. The chemical treatment aims to selectively eliminate only undesirable plants or insects. However, physical pest control is time consuming, chemical pest control can have negative side effects on the environment generally, and both are relatively costly.

### SUMMARY

In accordance with a broad aspect, there is provided an autonomous unmanned ground vehicle (AUGV). The AUGV comprises: a chassis with a drive mechanism to displace the AUGV among a plurality of plants, the plurality of plants comprising at least one weed; an image-capture device mounted to the chassis to obtain images of the plurality of plants; a motorized arm extending between a proximal end mounted to the chassis and a distal free end, the free end of the arm being displaceable with respect to the chassis; a microwave emitter mounted to the free end of the arm and displaceable therewith, the microwave emitter being operable to emit microwaves; and a control system to operate at least the drive mechanism, the image-capture device, the motorized arm, and the microwave emitter. The control system comprises a processing unit; and a non-transitory computer-readable memory having stored thereon instructions executable by the processing unit for causing the AUGV to perform at least: independently navigating the chassis among the plurality of plants; identifying the at least one weed in the images obtained by the image-capture device; displacing the motorized arm to position the free end thereof in proximity to the at least one weed; and emitting microwaves from the microwave emitter toward the at least one weed.

In some embodiments, the AUGV further comprises a lens affixed to the microwave emitter to focus the microwaves into a microwave beam, wherein emitting microwaves from the microwave emitter toward the at least one weed comprises directing the microwave beam toward the at least one weed.

In some embodiments, the lens is composed of paraffin.

In some embodiments, the instructions cause the AUGV to identify the at least one weed using a machine-learning algorithm.

In some embodiments, the instructions cause the AUGV to use at least one neural network stored in the memory.

In some embodiments, the instructions cause the AUGV to identify the at least one weed by: transmitting, over a data connection, at least some of the images to a remote server; and receiving, over the data connection, an indication of the at least one weed in the images from the remote server.

In some embodiments, the instructions cause the AUGV to identify at least one target area of the at least one weed, and to emit microwaves toward the at least one target area of the at least one weed.

In some embodiments, the image-capture device comprises at least one visible-light camera and at least one infrared camera.

In some embodiments, the instructions cause the AUGV to independently navigate the chassis among the plurality of plants by: determining, via a global positioning system, a current location of the chassis; identifying a location of interest within a predetermined area in which the plurality of plants are located; determining a course from the current location of the chassis toward the location of interest; and navigating the chassis to the location of interest.

In some embodiments, the AUGV further comprises a battery, wherein the instructions are further executable for causing the AUGV to perform: determining a charge level of the battery; comparing the charge level to a predetermined threshold; and when the charge level is below the predetermined threshold, independently navigating the chassis to a charging station.

In accordance with another broad aspect, there is provided a method for performing weeding among a plurality of plants, comprising: autonomously navigating a vehicle among the plurality of plants; obtaining images of the plurality of plants; identifying at least one weed in the images of the plurality of plants; displacing a motorized arm to position a free end thereof in proximity to the at least one weed; and emitting microwaves from the free end of the arm toward the at least one weed.

In some embodiments, emitting microwaves comprises focusing the microwaves into a microwave beam and directing the microwave beam toward the at least one weed.

In some embodiments, identifying the at least one weed comprises using a machine-learning algorithm to identify the at least one weed.

In some embodiments, using the machine-learning algorithm comprises using at least one neural network.

In some embodiments, identifying the at least one weed comprises: transmitting, over a data connection, at least some of the images to a remote server; and receiving, over the data connection, an indication of the at least one weed in the images from the remote server.

In some embodiments, identifying the at least one weed comprises identifying at least one target area of the at least one weed, and wherein emitting microwaves comprises emitting microwaves toward the at least one target area of the at least one weed.

In some embodiments, autonomously navigating the vehicle comprises: determining, via a global positioning system, a current location of the vehicle; identifying a location of interest within a predetermined area in which the plurality of plants are located; determining a course from the current location of the vehicle toward the location of interest; and displacing the chassis autonomously to the location of interest.

In some embodiments, autonomously navigating a vehicle among the plurality of plants comprises providing the vehicle with geospatial data indicative of the predetermined area, wherein the geospatial data includes a plurality of waypoints for navigating the vehicle, wherein the waypoints are produced by artificial intelligence.

In some embodiments, the method further comprises: determining a charge level of a battery powering the vehicle; comparing the charge level to a predetermined threshold; and when the charge level is below the predetermined threshold, independently navigating the vehicle to a charging station.

In some embodiments, autonomously navigating a vehicle among the plurality of plants comprises navigating between rows of the plurality of plants in a field, wherein the plurality of plants are disposed on opposite sides of the vehicle.

In accordance with a further broad aspect, there is provided a system for performing weeding among a plurality of plants. The system comprises: an AUGV; and at least one server communicatively coupled to the AUGV and configured for receiving, over a data connection, at least some of the images from the AUGV and for transmitting, over the data connection, an indication of the at least one weed to the AUGV.

In accordance with a still further broad aspect, there is provided a system for performing weeding among a plurality of plants, comprising: a plurality of AUGVs; and instructions causing the AUGVs to autonomously synchronize navigating, identifying, displacing, and emitting

There is also disclosed a handheld weeding device, comprising: an elongated shaft having a first end and a second end; a power connector attached to the shaft; a radiation source attached to the shaft and electrically connected to the power connector; and an actuator mechanism attached to the shaft between the first and second end and electrically connected to the radiation source for selectively controlling production of radiation by the radiation source in response to actuation of the actuator mechanism.

In some embodiments, the power connector is electrically connectable to an electrical cable providing electrical power to the handheld weeding device.

In some embodiments, the power connector is electrically connectable to a power source providing electrical power to the handheld weeding device.

In some embodiments, the radiation source comprises a microwave source.

In some embodiments, the microwave source comprises a magnetron.

In some embodiments, the handheld weeding device further comprises a waveguide disposed proximate the second end of the shaft and sized for performing beamforming of the radiation produced by the microwave source.

In some embodiments, the radiation source comprises an radio-frequency radiation source.

In some embodiments, the handheld weeding device further comprises an antenna disposed within the shaft proximate the second end and electrically connected to the radio-frequency radiation source for directing the radiation produced by the radio-frequency radiation source outward from the second end of the shaft.

In some embodiments, the handheld weeding device further comprises a hollow member attached to the shaft at the second end, the hollow member sized for substantially covering a weed.

In some embodiments, the actuator mechanism comprises a trigger.

In some embodiments, the handheld weeding device further comprises a handle member attached to the shaft between the first end and the second end.

In some embodiments, the handheld weeding device further comprises an orientation sensor coupled to the shaft and electrically connected to the radiation source, wherein the orientation sensor is operable to selectively deactivate the radiation source based on an orientation of the handheld weeding device.

In some embodiments, the handheld weeding device further comprises an imaging device coupled to the shaft proximate the second end and electrically connected to the radiation source, wherein the imaging device is operable to: acquire at least one image of a region to which the radiation from the radiation source would be applied following actuation of the actuator mechanism; and selectively deactivate the radiation source based on the acquired image.

### BRIEF DESCRIPTION OF THE DRAWINGS

The invention will be described in greater detail with reference to the accompanying drawings, in which:

FIG. 1A is a top-down view of an example field of crops containing weeds in which an example autonomous unmanned ground vehicle (AUGV) can operate;

FIG. 1B is a side view of the example AUGV of FIG. 1A in the example field of crops;

FIG. 2A is perspective view of an example autonomous unmanned ground vehicle (AUGV) in accordance with an embodiment;

FIG. 2B is perspective view of an example autonomous unmanned ground vehicle (AUGV) in accordance with another embodiment;

FIG. 3A is a perspective view of a motorized arm of the AUGV of FIG. 2A;

FIG. 3B is a perspective view of a motorized arm of the AUGV of FIG. 2B;

FIG. 4 is a perspective view of the underside of the AUGV of FIG. 2A;

FIG. 5 is a block diagram of an example control system for operating the AUGV of FIG. 2A or 2B;

FIG. 6 is side view of the AUGV of FIG. 2A in an example charging station;

FIG. 7 is a block diagram illustrating an embodiment of a computing system for implementing the control system of FIG. 5 in accordance with an embodiment described herein;

FIGS. 8A-B are flowcharts illustrating methods for performing pest control among a plurality of plants in accordance with an embodiment;

FIG. 9A is a perspective view of a handheld weeding device; and

FIG. 9B is a perspective view of another handheld weeding device.

### DETAILED DESCRIPTION

Farmers and crop producers must perform various types of pest control when seeking to maximize crop growth and yield. One particular type of pest is undesirable plants, which can include weeds 120 and cryptogams, as well as diseased crops or once-desirable plants which are no longer desirable, for instance due to being unable to produce crops. Disclosed herein is an autonomous unmanned ground vehicle (AUGV) for performing pest control and an associated method for pest control.

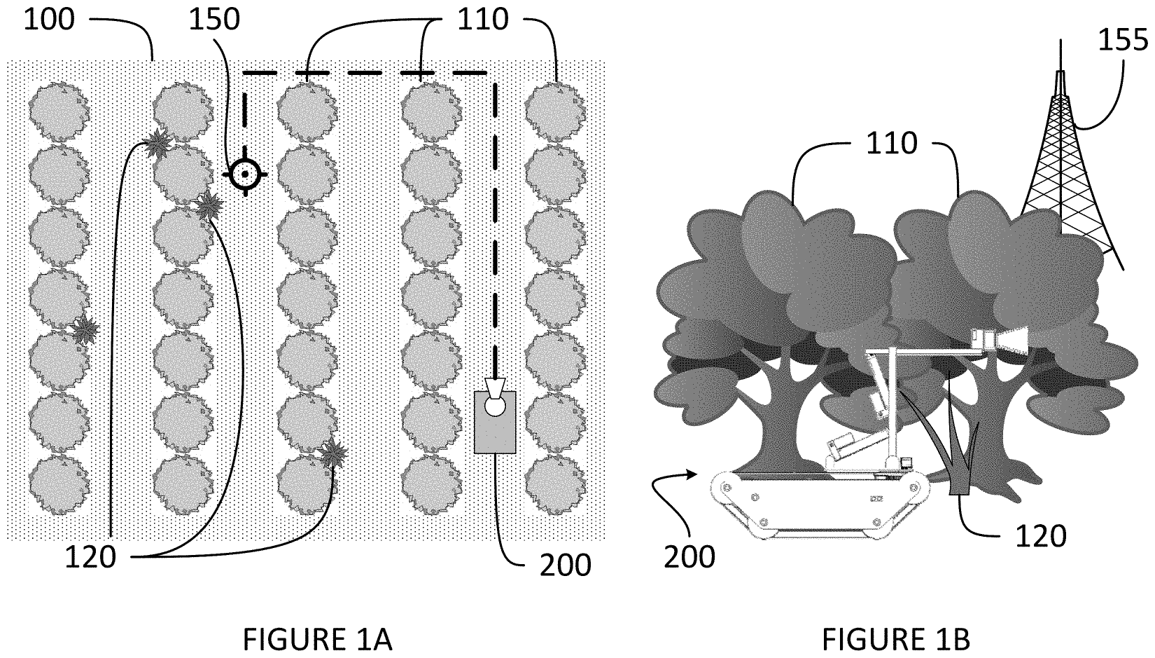

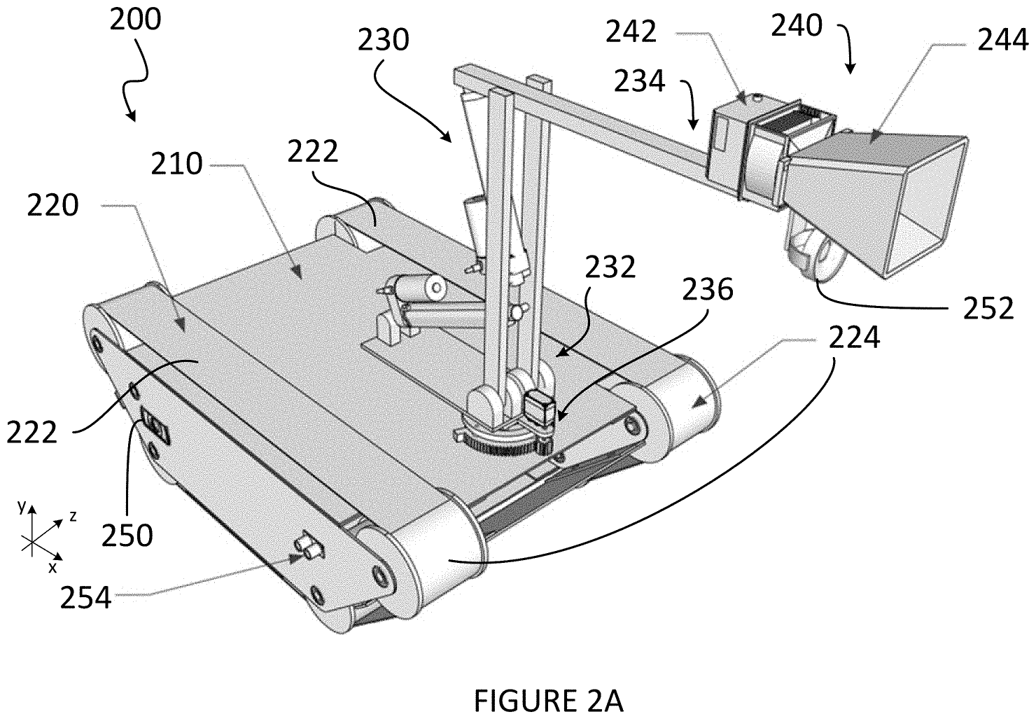

With reference to FIGS. 1A-B, an AUGV 200 is sized for autonomously patrolling through a field 100 or other terrain where crops 110 are located, for example between rows of crops and toward a target location 150, and is programmed to automatically detect and eliminate undesirable plants, such as weeds 120, via directed microwave emission. In some embodiments, the AUGV is used in vineyards and berry farms. Although the discussion herein primarily focuses on weeding, which is the act of eliminating weeds 120 or other undesirable plants, it should be noted that similar techniques to those disclosed herein can be applied to other types of pest control, including the elimination of insects and small animals, under certain conditions. For example, in an embodiment, the techniques disclosed herein are applicable to insects and/or small animals which are immobile or substantially immobile for certain lengths of time. Additionally, though the discussion herein focuses on outdoor use, in which the AUGV 200 patrols the field 100 where crops 110 are located, it should be understood that the AUGV 200 can also be used in a greenhouse setting, in outdoor plant nurseries, and for landscaping purposes at private dwellings or buildings. In some embodiments, the AUGV 200 is configured for communicating with a central control 155, which can issue directives to the AUGV 200, receive information therefrom, and the like.

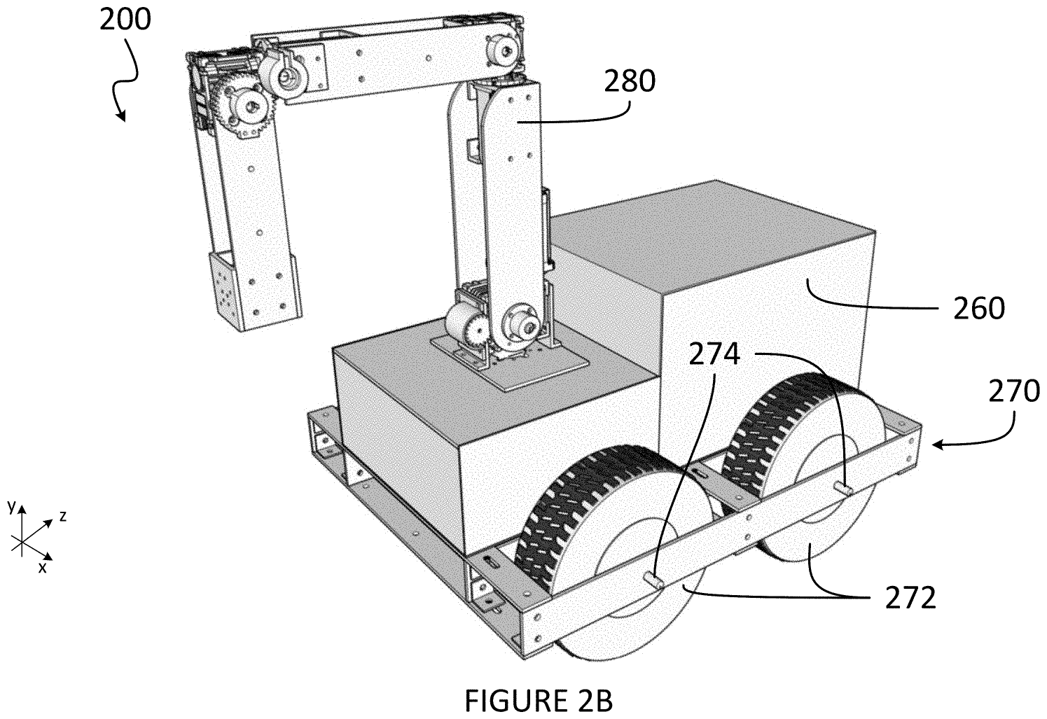

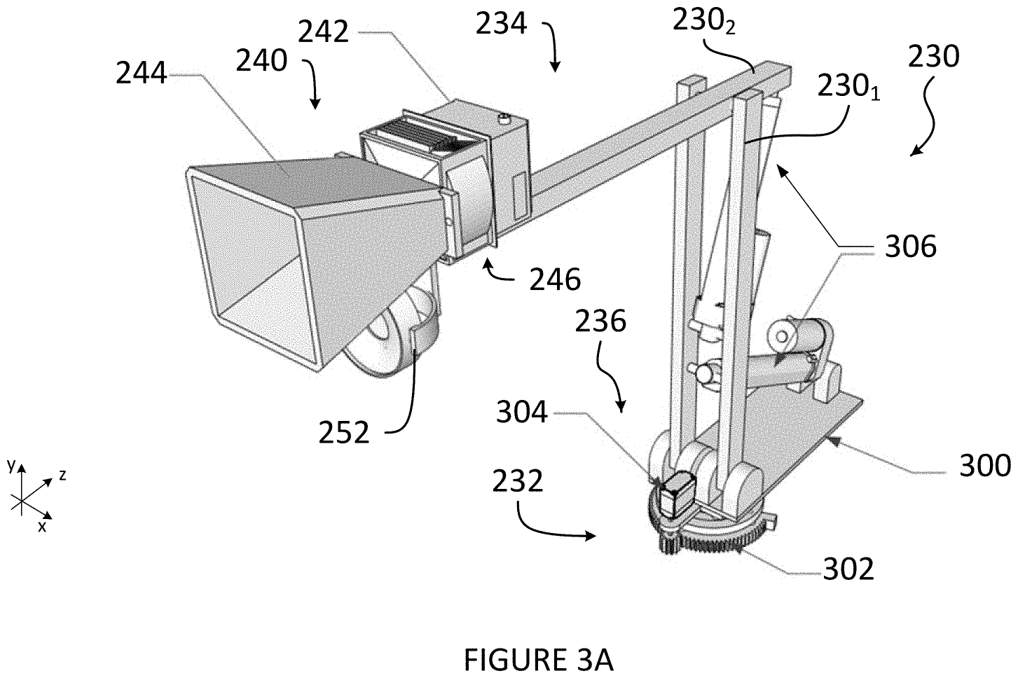

With reference to FIG. 2A, there is shown a perspective view of the AUGV 200. The AUGV 200 is composed of a chassis 210 to which is attached a drive mechanism 220 and a motorized arm 230. The chassis 210 can adopt any suitable shape and can be composed of any suitable material. In some embodiments, the chassis 210 is shaped as a rectangular prism, such as a trapezoidal prism, or has any other suitable shape. The chassis 210 can be made of any suitably resilient material, for example aluminium, titanium, steel, fiber glass, and hard plastics, such as thermoplastics, polycarbonates, polypropylenes, polyurethanes, and the like. In some embodiments, the chassis 210 is shaped like a tank or similar ground-based vehicle, although with smaller dimensions. For example, the chassis 210 is 84 cm long, 61 cm wide, and 41 cm tall. It should be understood, however, that the chassis 210 can adopt any dimensions suitable for patrolling between rows of crops 110 in the field 100, or between the crops 110 themselves. In addition, the chassis 210 can be designed to have a sufficiently low weight to allow for the AUGV 200 to be battery operated.

The drive mechanism 220 is attached to the chassis 210 and allows the AUGV 200 to be displaced across terrain. In some embodiments, the drive mechanism 220 is composed of a pair of endless tracks 222 which rotate over wheel hubs 224. The wheel hubs 224 guide the movement of the endless tracks 222. In some cases, the endless tracks 222 are provided with treads or other gripping elements thereon for improving traction between the endless tracks 222 and the ground. Still other types of endless tracks 222 are considered. In other embodiments, the drive mechanism 220 is composed of one or more wheels, which can be inflatable, solid, collapsible, spring-based, and the like. In still further embodiments, the drive mechanism 220 is composed of a plurality of displaceable legs or posts. Still other embodiments are considered.

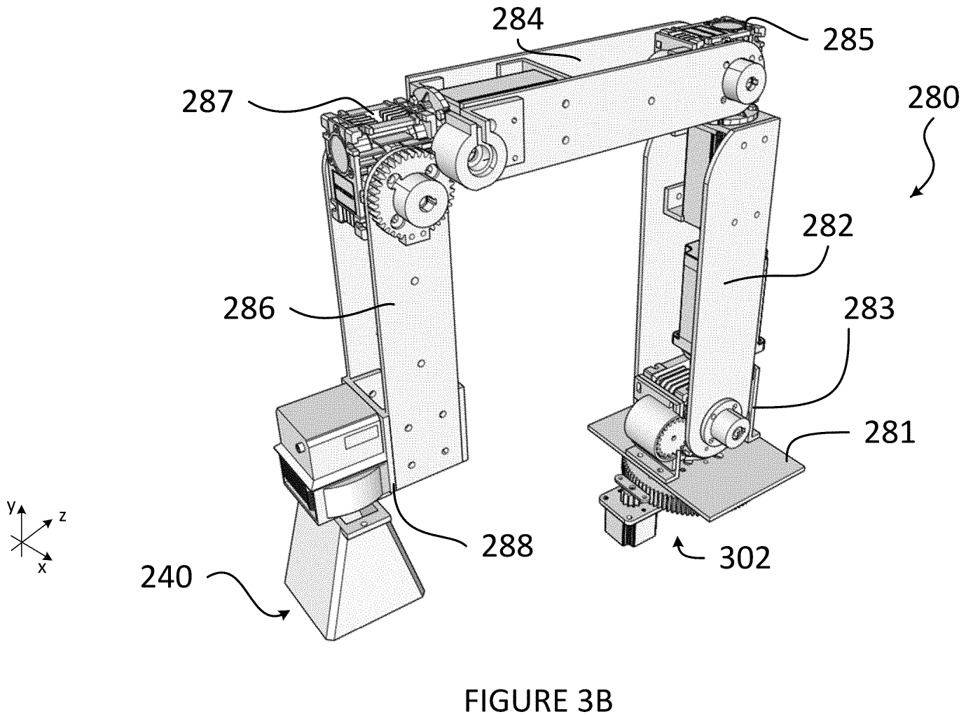

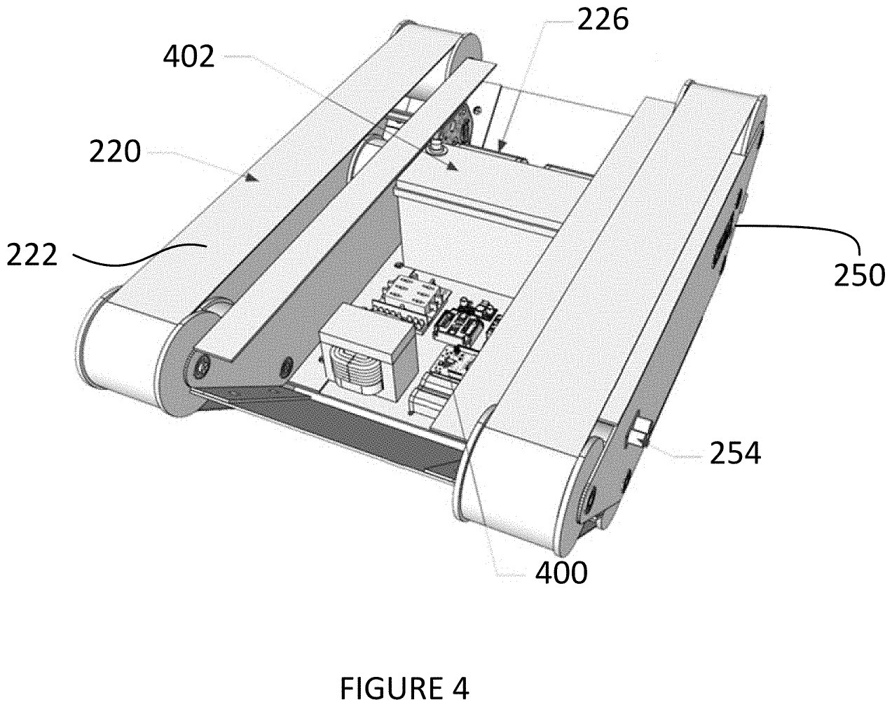

With additional reference to FIG. 3A, the motorized arm 230 has a proximal end 232 and a distal free end 234, and can be composed of a plurality of link members 230₁, 230₂, allowing the arm 230 to move in an articulated fashion. The proximal end 232 is affixed to the chassis 210 of the AUGV 200 via a fixture 236 and a base 300. The fixture 236 can include a turntable 302 or other rotating element to allow the arm 230 to rotate in a horizontal plane (x-z plane). For example, the turntable includes a gear bearing system which allows the arm 130 to rotate up to 180°, or any other suitable range. In addition, the fixture 236 can include a motorized hinge 304 or other rotating element to allow the arm to rotate in a vertical plane (x-y plane). In some embodiments, the arm 230 includes a plurality of actuators 306 to cause the various link members 230₁, 230₂ of the arm 230 to move in a variety of directions. The actuators 306 can include pistons, motors, and the like.

At the free end 234 of the arm 230 is mounted a microwave emitter 240 which is used to eliminate pests, for example weeds 120. The microwave emitter 240 is composed of a microwave source 242 and a waveguide 244. The microwave source 242 uses a maser, a magnetron, or other device suitable for producing microwaves, and which can be adjusted so that the microwaves produced are suitable for causing dielectric heating in the weeds 120. For example, the microwave source 242 produces microwaves in the range of 2.45 GHz to 10 GHz. Prolonged exposure to the microwaves can thus cause the weeds 120 to perish. The waveguide 244 is used to direct the microwaves toward the weeds 120. The waveguide can be made of any suitable material, for example aluminium, steel, titanium, and the like. In some embodiments, the waveguide 244 is shaped so that the waveguide 244 can be positioned to cover or encapsulate at least a portion of a weed 120 to be eliminated. In some embodiments, the microwave emitter 240 includes an articulated element allowing the microwave source 242 and/or the waveguide 244 to be positioned to cover or encapsulate at least a portion of the weed 120. In the depicted embodiment, the waveguide 244 has a cross-sectional area that increases in a direction away from the free end 234 of the arm 230.

In addition, in some embodiments the microwave emitter 240 comprises a lens 246 for focusing the microwaves produced by the microwave source 242 into a microwave beam. In the depicted embodiment, the lens 246 is made of paraffin and serves to reduce dissipation of the emitted microwaves. The microwave source 242 and the lens 246 can be positioned relative to one another so that the microwaves emitted from the microwave source 242 reach the lens 246 at a predetermined angle. The focused microwave beam can be more easily directed by the microwave emitter 240, for example via the waveguide 244, toward a particular target area or weed 120, and can allow the microwave emitter 240 to direct microwaves at weeds 120 which are further from the arm 230. In some embodiments, the lens 246 is selected with a particular focal length, for instance 75 cm, to standardize the position of the arm 130 relative to the weed 120.

For example, if the stem of the weed 120 is particularly vulnerable to microwaves, the waveguide 244 directs the microwave beam toward the stem of the weed 120. In another example, an effective way of eliminating a particular type of weed 120 is to apply microwaves to the roots of the weed 120, and the waveguide 244 directs the microwave beam theretoward. Still other use cases are considered. For example, in some cases a fluid applicator is also attached to the free end 234, which is configured for applying or spraying a fluid, for instance oil, to the weed 120 prior to or during production of microwaves. The fluid can accelerate the rate at which the weed 120 is heated via the microwaves or reduce the amount of microwaves required for the weed 120 to be eliminated. For example, the weed 120 can be sprayed with microdots prior to emitting the microwave beam thereat. In another example, the amount of time during which the microwaves are directed at the weed 120 is varied based on the type of weed, the size of the weed, the stage of the growth cycle of the weed, and the like.

Although the disclosure above focuses primarily on microwave-based elimination of weeds 120, other embodiments of the AUGV 200 could substitute, or supplement, the microwave emitter 240 with a similar laser-based system. For example, a carbon dioxide laser, or any other suitable laser, can be used. Alternatively, or in addition, the microwave emitter 240 could be substituted for, or supplemented with, a device for producing radio waves and/or sound waves suitable for damaging or eliminating weeds. Still other approaches are considered.

Returning to FIG. 2A, the AUGV 200 additionally comprises an image-capture device 250. In the embodiment shown in FIG. 2A, the image-capture device 250 is a wide-angle camera mounted on a side of the chassis 210 which provides the AUGV 200 with vision of crops 110 as the AUGV 200 patrols through rows of crops 110 in the field 100. Nevertheless, it should be understood that the image-capture device 250 can include any number of cameras which can be mounted at any suitable location on the chassis 210 and/or on the arm 230. In addition, the image-capture device 250 can include a light source 252 to allow the image-capture device 250 to function in low-light conditions, for example a lamp, which can be used at night, in fog or rain, and the like.

In some embodiments, the image-capture device 250 includes one or more visible-light cameras, one or more infrared cameras, one or more ultraviolet cameras, and the like. For example, the image-capture device 250 includes two wide-angle visible light cameras, each mounted on an opposing side of the chassis 210, and a combination camera mounted to the free end 234 of the arm 230, for instance with the waveguide 244, which is configured for capturing infrared, visible, and ultraviolet light. Still other embodiments are considered. For example, a light detection and ranging (LIDAR) or radio detection and ranging (RADAR) system can be used for navigation and/or for image acquisition. For instance, the AUGV 200 includes one or more ultrasonic sensors 254. In another example, certain pests may be reactive to ultraviolet light, for instance cryptogams, insect eggs, and the like. Thus, an ultraviolet light source can be included as part of the light source 252 affixed to the free end 234 of the arm 230 to assist in the identification of pests reactive to ultraviolet light.

The AUGV 200 is configured for acquiring images via the image-capture device 250 in order to detect and identify weeds 120 for elimination. As the AUGV autonomously patrols through terrain where crops are located, the image-capture device 250 collects images of the crops, and of any weeds 120 growing alongside the crops. The AUGV 200 is configured for identifying the weeds 120 in the images captured and then for eliminating the weeds 120.

As used herein, the term “autonomously” is understood to mean substantially without real-time human direction. Thus, the AUGV 200 can be said to operate in an autonomous fashion when it is given broad guidelines, for instance an area to patrol or a location to travel to, and then operates in accordance with those guidelines without real-time human direction controlling the operation of the AUGV 200. For example, an AUGV 200 can be given an predetermined area to patrol, which can include boundaries based on latitude and longitude, or a radius from a reference point. When operation of the AUGV 200 is commenced, the AUGV 200 then determines by itself, i.e. without human intervention, what route to take, which locations within the predetermined area to reach first, and the like.

Although described herein as operating autonomously, the AUGV 200 may in addition to its autonomous operation, or as a substitute therefore, be operated remotely in response to human direction. The AUGV 200 may therefore have a remote operation mode which allows a human operator to control or pilot the AUGV 200 using a control device such as a laptop, tablet, smartphone, handheld console, remote controller, or other suitable device, including any suitable portable electronic device. The remote operation mode may allow the human operator to override the autonomous operation of the AUGV 200 in order to control one or more AUGVs 200 from a distance.

With reference to FIG. 2B, in an alternative embodiment, the AUGV 200 is composed of a chassis 260, a drive mechanism 270, and a motorized arm 280. The chassis 260 can be similar to the chassis 210, can be made of any suitable material and take on any suitable shape and size. The drive mechanism 270 is attached to the chassis 260 and allows the AUGV 200 to be displaced across terrain. In the embodiment of FIG. 2B, the drive mechanism 270 is composed of four wheels 272 disposed on two axles 274. The wheels 272 can be of any suitable size, and can be inflatable, solid, collapsible, spring-based, and the like. For example, the wheels can be 33 cm in diameter. In some embodiments, the wheels 272 are provided with treads or other gripping elements to allow the AUGV 200 to patrol various types of terrain. Other embodiments of the drive mechanism 270 can include more, or fewer wheels 272, as appropriate.

With additional reference to FIG. 3B, the motorized arm 280 can be an alternative embodiment to the arm 230. The arm 280 can be composed of a base 281 and three arm branches 282, 284, 286. The base 281, like the base 300, can include the turntable 302 or other rotating element to allow the arm 280 to rotate in a horizontal plane (x-z plane). The arm branch 282 is coupled to the base 281 at a first end, and to the arm branch 284 at a second end. The branch arm 284 is coupled between arm branches 282 and 286 at first and second ends thereof. The arm branch 286 is coupled to arm branch 284 at a first end, and defines a connector 288 at a second end, to which the microwave emitter 240 can be connected. Linking branches 282, 284, 286 are arm mechanisms 283, 285, 287, which can each be composed of various motors and gearboxes, to allow movement of the branches in one or more axes. Other embodiments of motorized arms, including with more or fewer branches, are also considered.

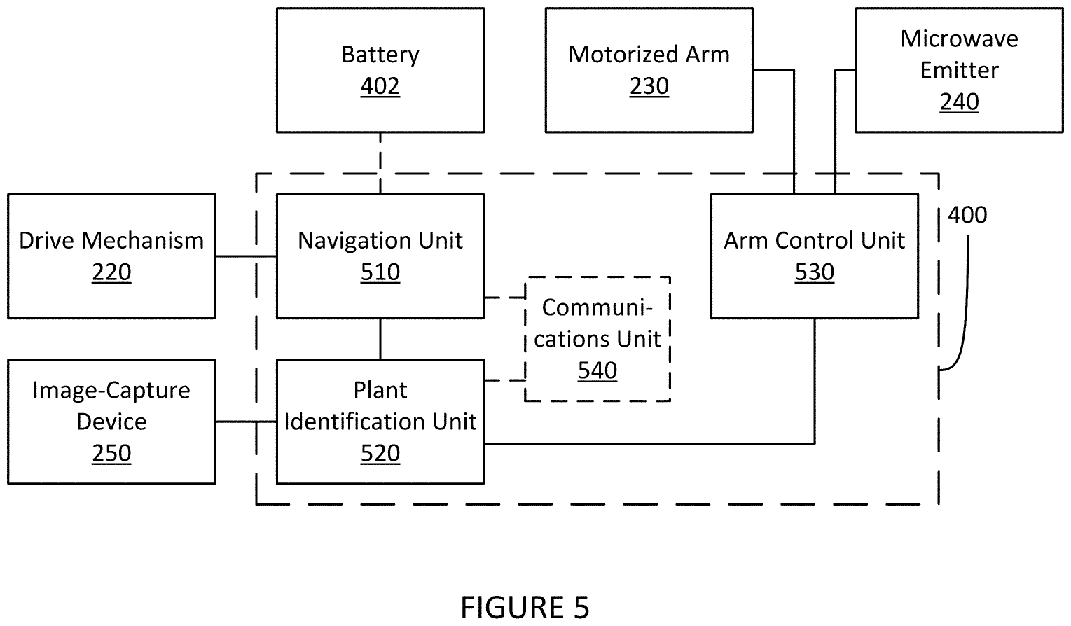

With reference to FIG. 4 operational control of the various components of the AUGV 200, including the drive mechanism 220, the motorized arm 230, the microwave emitter 240, and the image-capture device 250, is performed by a control system 400. The control system 400 can include any suitable electrical and electronic components for effecting operational control of the AUGV 200, including microcontrollers or other processing units, transformers, inverters, relays, breakers, fuse blocks, and the like. For example, to control operation of the drive mechanism 220, the control system 400 can control operation of a plurality of motors 226 which cause the endless tracks 222 to move, thereby imparting motion to the AUGV 200. The physical components of the control system 400 can be mounted to the chassis 210 and/or to the arm 230 of the AUGV 200 in any suitable fashion. In some embodiments, the physical components of the AUGV 200 are located in a substantially weatherproof compartment or enclosure, to ensure that the AUGV 200 is operational regardless of weather conditions.

In addition, the AUGV 200 includes a power source for powering the drive mechanism 220, the motorized arm 230, the microwave emitter 240, the image-capture device 250, and the control system 400. In the embodiment shown in FIG. 4, the AUGV 200 includes a battery 402, which provides electrical power to the AUGV 200. For instance, the battery 402 is selected to have sufficient electrical capacity to allow the AUGV 200 to operate for at least 30 min, at least 1 hour, at least 2 hours, or any other time period. In other embodiments, another power source can be used. For example, the AUGV 200 includes a gas engine, which powers the drive mechanism 220, and an alternator for providing electrical power to the motorized arm, the image-capture device 250, and the control system 400. Fuel for the gas engine can be stored in a fuel tank.

In a particular implementation, the battery 402 is a 12V, 200Ah battery. The battery 402 connects to a 120A breaker, which in turn is attached to a fuse block which splits wired connections to the various components of the AUGV 200. The fuse block has a plurality of 30A resettable fuses to which each of the components are connected. The motors 226 and the actuators 306 are controlled by motor controllers, which can be of any suitable brand or make. The motor controllers can be connected on separate lines to the fuse block. The rotatable elements of the fixture 236 can be controlled by a servo motor having a servo motor controller. In some cases, the gearing system of the turntable of the fixture 236 has a gearing system reduction ratio of 1:7 and a potentiometer sensor. The servo motor controller is also connected on a separate line to the fuse block. Since the servo motor for the fixture 236 operates at a different voltage than the motors 226 and the actuators 306, a buck inverter or other voltage divider system is used to supply the appropriate voltage to the servo motor.

Continuing with the particular implementation, power for the microwave emitter 240 is provided via a 12V inverter connected to the battery 402 which is used to produce a 120V output, which is attached to a transformer. Output wires from the transformer are applied to a doubling capacitor, which is then attached to the microwave source 242, in this implementation a magnetron located at the free end 234 of the arm 230. When power from the battery 402 is applied, the magnetron releases microwaves used to kill the weed 120. Although the particular implementation is illustrated in FIG. 4, it should be noted that this is only for the purpose of illustration, and that other implementations are considered.

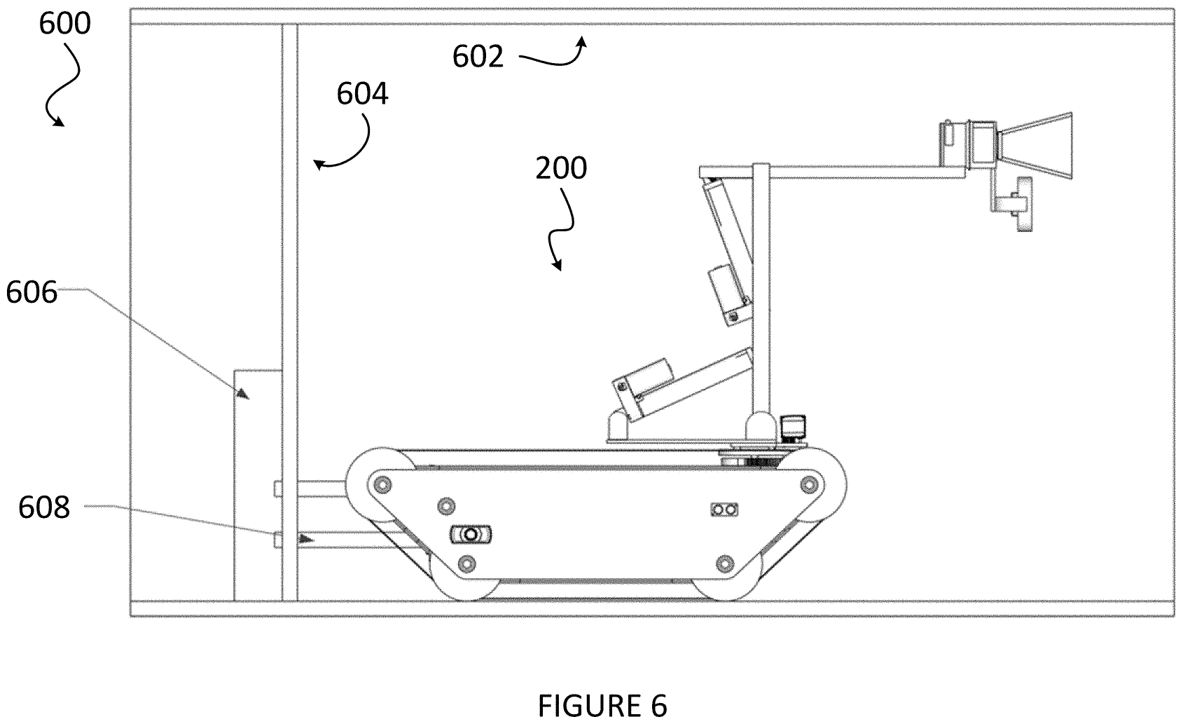

With reference to FIG. 5, a schematic representation of the control system 400 is shown. The control system 400 includes a navigation unit 510, a plant identification unit 520, an arm control unit 530, and optionally a communications unit 540. The control system 400 is configured for interacting with the drive mechanism 220, the motorized arm 230, the microwave emitter 240, and the image-capture device 250.

The navigation unit 510 is configured for independently navigating the chassis 210, and the AUGV 200 as a whole, among a plurality of plants, for example through a field or other predetermined area in which crops are located, for example by sending commands to the drive mechanism 220 to cause the drive mechanism to move the AUGV 200 in a particular fashion. In some embodiments, the navigation unit 510 accesses a global positioning system (GPS) or other navigation system, which can be part of the communications unit 540, to determine the position of the AUGV 200 at a particular time. For example, the AUGV 200 can be tasked with patrolling a predetermined area where the plurality of plants are located. The GPS is used to determine latitude and longitude coordinates for the AUGV 200, and the navigation unit 510 uses maps or other resources to locate the AUGV 200 within the predetermined area. The navigation unit 510 then identifies a location of interest to which the AUGV 200 should be navigated, determines a course toward the location of interest, for instance while taking into consideration the terrain in the predetermined area, and navigates the AUGV 200 to the location of interest.

In some embodiments, the navigation unit 510 can be provided with a map outlining a predetermined area to be patrolled, for example part or all of the field 100, in the form of a GeoTiff file or other suitable geospatial information. An operator of the AUGV 200 can plot waypoints, boundaries, and the like within the GeoTiff file, although in some embodiments an artificial intelligence system can be used to automatically or semi-automatically produce the GeoTiff file. For example, images of the field 100 can be acquired using drones or other unmanned aerial vehicles. The images are stitched together to form a map, and an operator of the AUGV 200 defines boundaries for the area to be patrolled by the AUGV 200. The artificial intelligence system can then automatically generate waypoints or other points of interest for the AUGV 200. The GeoTiff file is provided to the AUGV 200, which navigates the area defined within the GeoTiff file in accordance therewith.

In some embodiments, the navigation unit 510 also relies on visual landmarks to perform navigation of the AUGV 200. For example, images acquired via the image-capture device 250 can be used to identify landmarks or locations of interest. The navigation unit 510 can then be informed of the presence of landmarks and/or locations of interest, and the navigation unit 510 can command the drive mechanism 220 accordingly. In some embodiments, the AUGV 200 uses images captured by the image-capture device 250 to create a visual map of the field 100, and the navigation unit 510 can use the visual map to perform navigation of the AUGV 200. Additionally, other types of sensors, for instance the ultrasonic sensors 254, can be used for obstacle avoidance.

In addition, in some embodiments the navigation unit 510 is configured for communicating with a remote server, which can be part of the central control 155, or separate therefrom. Optionally, the communications unit 540 can include one or more cellular antennas, one or more GPS antennas, one or more WiFi antennas, and the like. The navigation unit 510 is configured for periodically and/or punctually reporting a position of the AUGV 200 to the remote server via the communications unit 540, for example over the Internet. In some embodiments, the navigation unit 510 reports the position of the AUGV 200 to the remote server every few seconds, every few minutes, every few hours, and the like. In other embodiments, the navigation unit 510 reports the position of the AUGV 200 in response to particular events, for example a weed 120 having been located and/or eliminated, or when the AUGV 200 malfunctions. In still other embodiments, the navigation unit 510 reports the position of the AUGV 200 when queried by a user at the remote server.

In addition, in some embodiments, the AUGV 200 is configured for acting in concert with other AUGV to collaboratively patrol the field 100. For example, the navigation unit 510 of the AUGV 200 can coordinate with the navigation unit of other AUGV 200, for example via the communications unit 540, or with the central control 155, to assign or receive assignments of routes to follow through the crops 110 in the field 100. In some instances, the AUGV also collectively coordinate to share charging stations, maintenance stations, and the like. For example, a first AUGV 200 is configured for patrolling the field 100 or a portion thereof and for acquiring images of the crops 110 located in the field 100. Once the first AUGV 200 has completed a particular patrol route, the first AUGV 200 can return to or contact the central control 155, upload all the images to a remote server for weed identification, and receive information pertaining to the location of weeds 120 along the patrol route. Then, the first AUGV 200, or another AUGV, can eliminate the weeds 120 identified along the patrol route.

In some embodiments, the navigation unit 510 can be informed, for example via the remote server, that a particular section of the predetermined area has a higher concentration of weeds 120 than other sections. The navigation unit 510 can thus patrol the particular section at a higher frequency than other sections of the predetermined area. In some instances, the AUGV 200 can patrol the particular section at a slower pace, or repeatedly traverse the particular section, or patrol the particular section along a variety of paths. Furthermore, in situations where multiple AUGVs 200 are used to collectively perform pest control in the predetermined area, the AUGVs 200 can coordinate via their respective communication units 540 to divide up the predetermined area and/or to collectively patrol the particular section with higher frequency.

The plant identification unit 520 is communicatively coupled to the image-capture device 250 for acquiring the images obtained by the image-capture device 250. The plant identification unit 520 is configured for identifying weeds 120 or other undesirable plants in the images obtained by the image-capture device 250. In some embodiments, the identification of weeds 120 by the plant identification unit 520 is performed onboard the AUGV 200, for example by the plant identification unit 520 itself. In other embodiments, the identification of weeds 120 is performed with assistance from a remote server or other resource, which informs the plant identification unit 520 when a weed is identified in the images acquired by the image-capture device 250. The plant identification unit 520 is configured for identifying weeds 120 or other pests at different life-cycle stages, with different morphologies, colouring, position, and the like.

In some embodiments, an artificial intelligence algorithm for weed identification. The artificial intelligence (AI) algorithm can be a traditional machine learning algorithm, a neural network, a deep learning algorithm, or any other suitable type of AI. In some embodiments, the AI algorithm may reside within the control system 400, for example in a computer-readable memory. In other embodiments, the plant identification unit 520 is configured for sending the images obtained by the image-capture device 250 to a remote server via the communications unit 540, where the AI is resident. The remote server can then indicate to the plant identification unit 520 when a weed 120 is identified. Once a weed 120 is identified, the plant identification unit 520 can instruct the navigation unit 510 and/or the arm control unit 530 to position the chassis 210 and/or the arm 230 to eliminate the weed 120.

For example, the Al can be trained using a database or other corpus of images of known undesirable plants, known insects, and the like. In some instances, the database includes images or other markers of undesirable plants at various stages of growth or morphology, in various lighting conditions, from various perspectives, and the like. In addition, the Al can be substantially continuously retrained based on newly acquired images, which can be validated by a human operator and/or a more complex AI, as appropriate.

In some embodiments, the plant identification unit 520 sends the images obtained by the image-capture device 250 to the remote server to train the AI used for weed identification. For example, the images can be reviewed by a human operator, or by another AI, and used as part of a corpus for AI training. In some instances, some or all images captured by the image-capture device 250 are sent to the remote server. In other instances, only images identified by the plant identification unit 520 as showing a weed 120 are sent. Still other approaches are considered. In some cases, the images sent to the remote server can also be used to monitor an overall health level of the crops and/or to monitor population levels of weeds 120, diseased crops, insects and other pests, and the like.

The arm control unit 530 is communicatively coupled to the motorized arm 230 for controlling operation thereof. For example, when the plant identification unit 520 instructs the arm control unit 530 that a weed 120 has been identified, the arm control unit 530 is configured to displace the arm 230 to position the free end 234 of the arm 230, where the microwave emitter 240 is located, in proximity to the at least one weed 120.

The arm control unit 530 is also communicatively coupled to the microwave emitter 240 for causing the microwave emitter 240 to emit microwaves toward the weed 120 once the arm 230 is appropriately positioned. In embodiments in which the microwave emitter 240 includes the lens 246, the arm control unit 530 can be configured to identify a target area of the weed 120 and to position the arm 230 so that the target area is exposed to the microwave beam produced by the microwave emitter 240.

The communications unit 540 is configured for communicating with the central control 155, with one or more remote servers, other AUGV, and the like, in accordance with the embodiments described hereinabove. The communications unit 540 can include one or more of cellular radios, antennas for Bluetooth™, Zigbee™, WiFi, and the like, or any other suitable communications means. In some embodiments, the communications unit 540 is configured for continuously, or punctually, reporting information about the AUGV 200 to the central control 155. For example, the AUGV 200 can report information about patrol paths, weed locations, a number of weeds eliminated, and the like, to the central control 155. The central control 155 can be configured to use the information reported by the AUGV 200 for statistical analysis to measure various characteristics pertaining to the field 100, the crops 110, the weeds 120, and the like. For example, weed growth patterns, crop health, etc. can be analyzed using the information provided by the AUGV 200.

In some embodiments, the control system 400, for example the navigation unit 510, is also configured for monitoring a level of the battery 402, indicative of a remaining charge in the battery 402. When the battery level falls below a predetermined threshold, the navigation unit 510 can independently navigate the AUGV 200 to a predetermined location for recharging of the battery 402. In some embodiments, the threshold is a fixed value, for example 20%, 25%, 30%, or any other suitable charge level of the battery 402. In other embodiments, the threshold is based on a distance between the current location of the AUGV 200 and the nearest charging station. In embodiments where other power sources are used, for example a gas engine, the navigation unit 510 is configured for monitoring a relevant indication of remaining operating time of the AUGV 200, for example a fuel level.

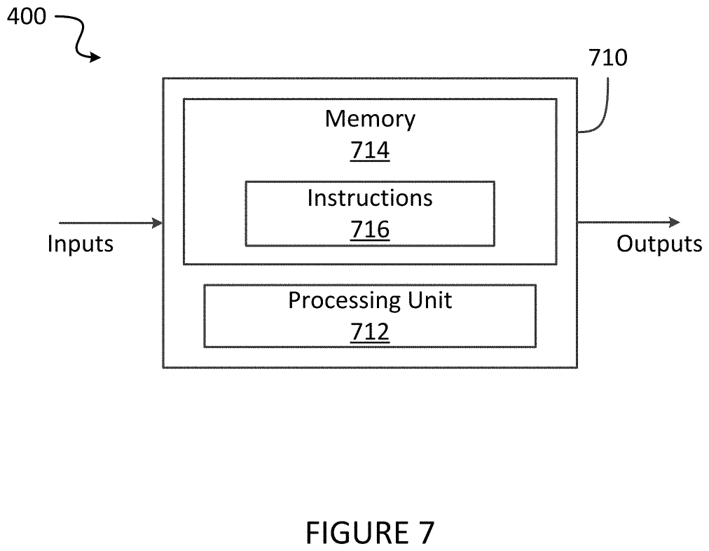

With reference to FIG. 6, an example charging station 600 for the AUGV 200 powered by the battery 402 is shown. The charging station 600 has a roof 602 and at least one wall 604 to protect the AUGV 200 from inclement weather when charging. The charging station 600 also includes a power source 606 and one or more connectors 608. The power source 606 produces electrical power for recharging the battery 402, which is provided to the AUGV 200 via the connectors 608. The electrical power can be provided as alternating current or direct current, and be provided at any suitable voltage and/or current level. The connector 608 can be configured for contacting portions of the chassis 210 of the AUGV 200 which feature charging pads to apply the electrical power to the battery 402, as appropriate. In a particular embodiment, the connector 608 includes four contact pads which correspond to four receiving pads on the chassis 210, which collaborate to form connecting pairs of contact pads and receiving pads. For example, three of the pairs are for charging the battery 402, and the fourth pair is for data communication between the charging station 600 and the battery 402.

Because the AUGV 200 is configured for automatically navigating to the charging station 600 to recharge the battery 402 when necessary, the AUGV 200 is fully autonomous and capable of substantially continuous operation. In addition, due to the weatherproof design of the chassis 210 and the ability of the image-capture device 250 to be used to identify weeds irrespective of lighting conditions and weather, the AUGV 200 can be deployed irrespective of environmental conditions. The use of directed microwaves to perform weeding is also a much more ecologically-friendly approach than traditional techniques, which often involve pesticides or other chemical agents. In addition, the AUGV 200 can be used to collect other information about the crops 110 and/or the field 100, based on the images acquired by the image-capture device 250. This information can be used to analyze crop success, weed distribution, and the like.

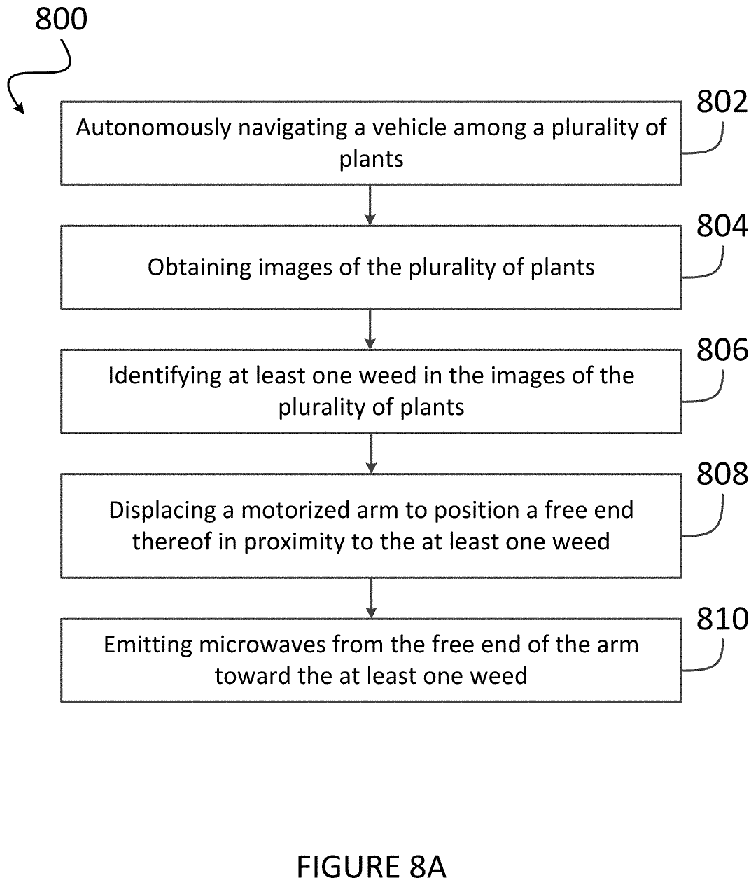

With reference to FIG. 7, the control system 400 may be implemented by a computing device 710, comprising a processing unit 712 and a memory 714 which has stored therein computer-executable instructions 716.

The processing unit 712 may comprise any suitable devices configured to implement the method 200 such that instructions 716, when executed by the computing device 710 or other programmable apparatus, may cause the functionality of the control system 400 described herein to be implemented. The processing unit 712 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 714 may comprise any suitable known or other machine-readable storage medium. The memory 714 may comprise non-transitory computer readable storage medium, for example, but not limited to, an electronic, magnetic, optical, electromagnetic, infrared, or semiconductor system, apparatus, or device, or any suitable combination of the foregoing. The memory 714 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 714 may comprise any storage means (e.g., devices) suitable for retrievably storing machine-readable instructions 716 executable by processing unit 712.

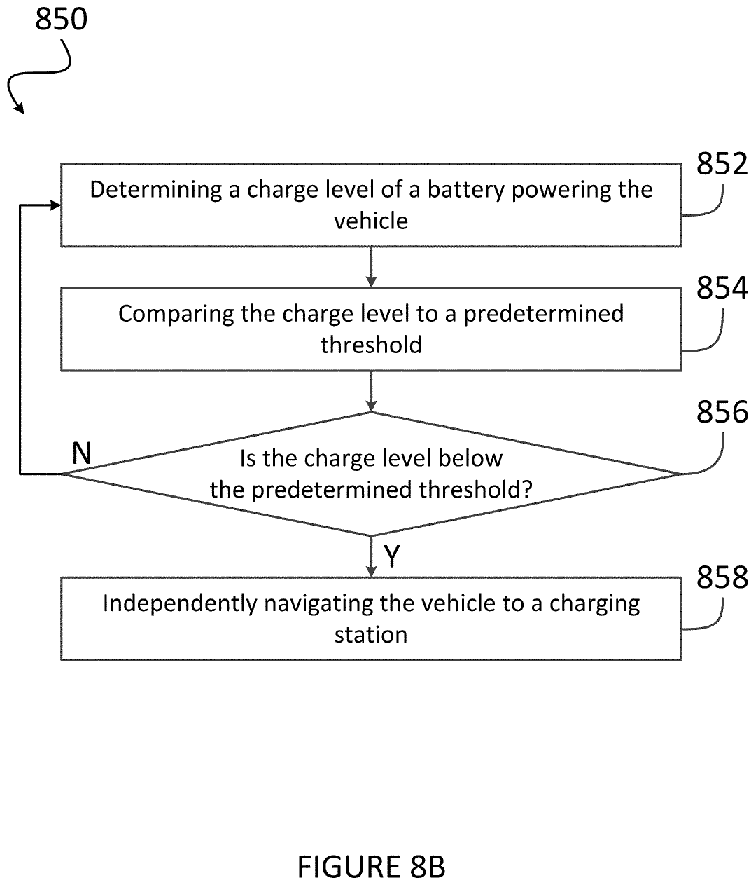

With reference to FIG. 8A, in some embodiments the control system 400 can implement a method 800 for pest control. At step 802, a vehicle, for example the AUGV 200, is autonomously navigated among a plurality of plants, for example in a predetermined area. At step 804, images of the plurality of plants are obtained, for example with the image-capture device 250. At step 806, at least one weed 120 is identified in the images. At step 808, a motorized arm is displaced to position a free end thereof in proximity to the at least one weed 120, for example the free end 234 of the arm 230. At step 810, microwaves are emitted from the free end 234 of the arm 230, for example via the microwave emitter 240, toward the at least one weed 120. The method 800 can be performed to eliminate or substantially damage the weed 120.

With reference to FIG. 8B, in some embodiments the control system 400 can additionally, or in the alternative, implement a method 850. At step 852, a charge level of a battery powering the vehicle, for example the battery 402, is determined. At step 854, the charge level of the battery 402 is compared to a predetermined threshold. At decision step 856, if the charge level is not below the predetermined threshold, the method 850 returns to some previous step, for example step 852. If the charge level is below the predetermined threshold, the method 850 moves to step 858. At step 858, the vehicle is independently navigated to a charging station, for example the charging station 600.

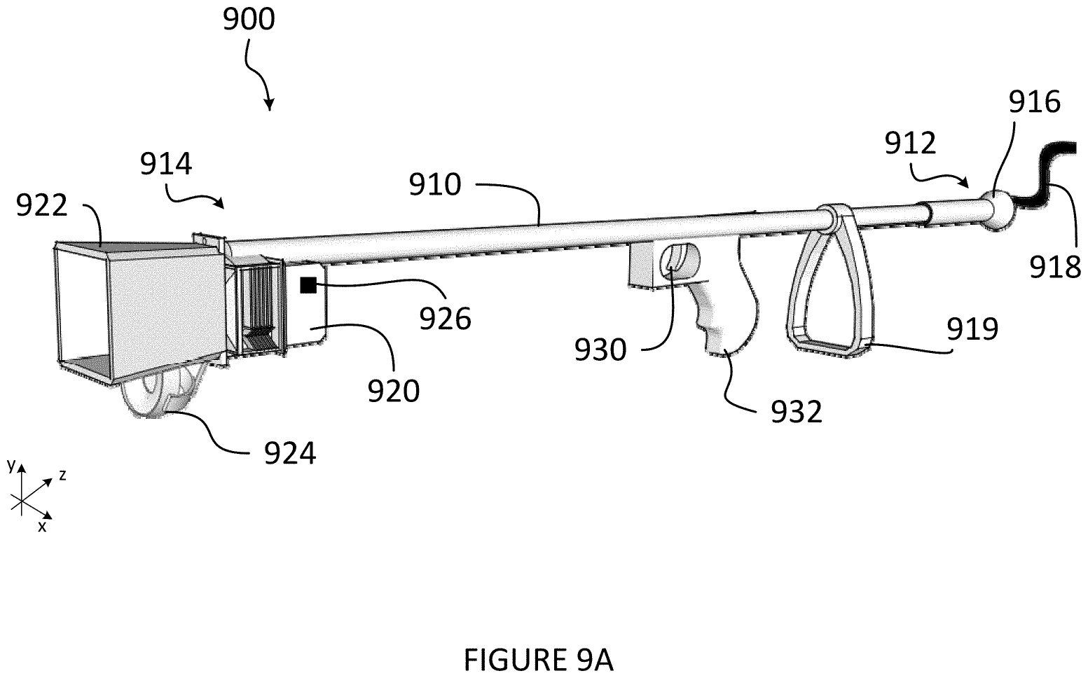

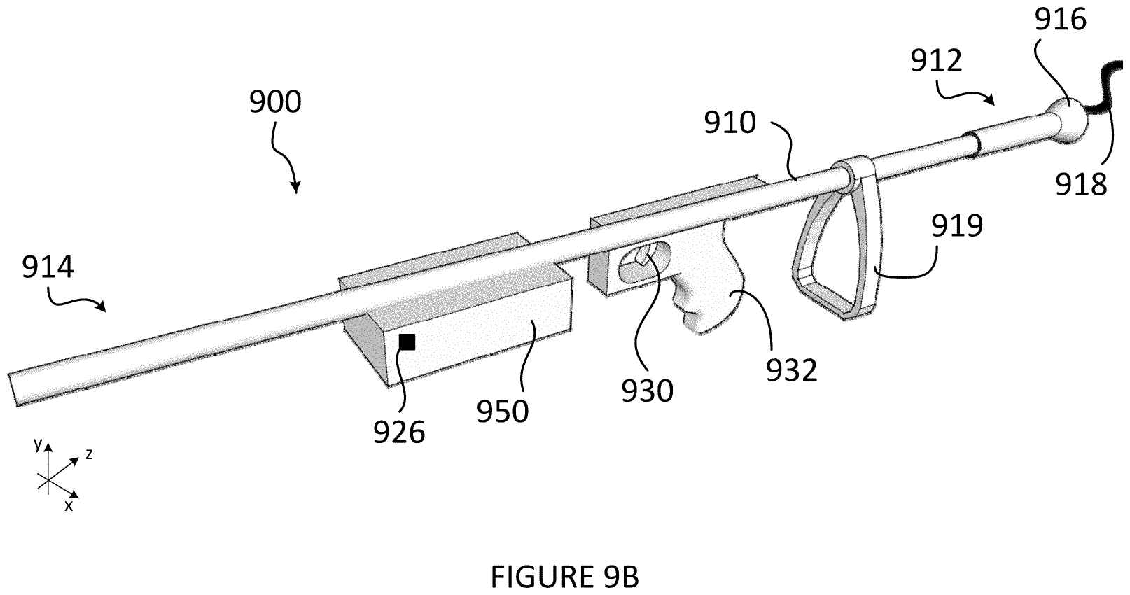

With reference to FIG. 9A, an alternative embodiment of at least some of the techniques provided by the present disclosure is illustrated, in the form of a handheld weeding device 900. The handheld weeding device 900 is composed of an elongated shaft 910, having first and second ends 912, 914, a radiation source 920, and an actuator mechanism 930. The shaft 910 can be of any suitable length and size, and can be cylindrical, prismatic, or any other suitable shape. In some embodiments, part or all of the shaft 910 is hollow, thereby defining an interior cavity to the shaft 910. In some embodiments, the shaft 910 has attached thereto a handle 919, for instance to facilitate handling of the weeding device 900. The handle 919 may also serve as a brace for supporting an arm.

Attached to the shaft is a power connector 916, which serves as a conduit for electrical power, for instance to supply the radiation source 920. The power connector 916 can be any suitable type of connector, for example a North American Manufacturers Association (NEMA) 1-15 or NEMA 5-15 connector. In some embodiments, the power connector 916 includes a power cable 918, which is used to link the power connector 916 to an exterior power source, such as a battery, mains power, or any other suitable source. In some other embodiments, the power connector 916 is configured for connecting to a portable power source, such as a battery, which can be affixed to the weeding device 900, for instance to the shaft 910.

The radiation source 920 serves to produce radiation used for the elimination of pests, such as weeds. In some embodiments, the radiation source 920 produces microwaves using a magnetron, for instance in the range of 2.45 GHz to 10 GHz. In other embodiments, the radiation source 920 produces a laser beam. In still further embodiments, the radiation source 920 produces a different kind of radiation suitable for eliminating weeds. The radiation source 920 is positioned to emit radiation along the same axis as the shaft 910, for example outward from the perspective of the second end 914. In some embodiments, the radiation source 920 has coupled thereto a waveguide 922 or other beam-forming element for constraining the way in which the radiation is produced. For example, the waveguide 922 can be a substantially hollow member sized for substantially covering a weed, so that radiation produced by the radiation source 920 is contained within the hollow member and applied substantially only to the weed. Other approaches are also considered.

The radiation source 920 can be attached to the shaft 910 in any suitable fashion, and can be disposed thereon at any suitable location. In some embodiments, the power connector 916 is disposed proximate the first end 912 of the shaft 910, and the radiation source 920 is disposed proximate the second end 914. Other embodiments are also considered. The radiation source 920 is coupled to the power connector 916, which provides electrical power to the radiation source 920. For example, the power connector 916 can include a cable for transferring electrical power obtained via the power cable 918 to the radiation source 920.

The actuator mechanism 930 serves to selectively control the production of radiation by the radiation source 920. In response to actuation of the actuator mechanism 930, the radiation source 920 is caused to produce radiation, for example by providing power to the radiation source 920. For example, the actuator mechanism 930 can include a switching element which becomes closed when the actuator mechanism 930 is actuated, thereby permitting the flow of electrical power from the power connector 916 to the radiation source 920. In some embodiments, the actuator mechanism 930 includes a trigger, for instance as illustrated in FIG. 9A. In other embodiments, the actuator mechanism 930 can include a button, a dial, a switch, or other similar device. The actuator mechanism 930 can be partially or substantially enclosed by a handle 932 or other member, for instance to facilitate handling of the weeding device 900 and access to the actuator mechanism 930. Additionally, the handle 932 can serve to reduce the risk of accidental actuation of the radiation source 920.

Other safety mechanisms can be integrated to the weeing device 900. For example, an image acquisition device 924 can be affixed to the radiation source 920 and/or to the waveguide 922. The image acquisition device 924 can be used to inspect an area to which radiation would be applied if the radiation source 920 was actuated. If the image acquisition device 924 detects that the area contains a weed or similar pest, the image acquisition device 924 can permit operation of the radiation source 920. Alternatively, if the image acquisition device 924 detects that the area contains an object which is not identified as a weed or similar pest, the image acquisition device 924 can interrupt operation of the radiation source 920. For example, the image acquisition device 924 can be configured for detecting the colour of objects in the area to which radiation would be applied. If the area is not substantially green, the image acquisition device 924 may prevent operation of the radiation source 920. In another example, the image acquisition device 924 can be configured for detecting heat signatures, and if the area contains a heat signature indicative of a human body part, such as a foot or a hand, the image acquisition device 924 can prevent actuation of the radiation source 920. Other approaches for using the image acquisition device 924 to selectively prevent or permit operation of the radiation source 920 are also considered.

Alternatively, or in addition, the weeding device 900 can include an orientation sensor 926, which can detect the orientation of the weeding device 900. The orientation sensor 926 can be configured for selectively preventing or permitting operation of the radiation source 920 depending on the orientation of the weeding device 900. For example, the orientation sensor 926 can permit operation of the radiation source 920 only when the orientation of the weeding device 900 is substantially upright. In another example, the orientation sensor 926 can prevent operation of the weeding device 900 when the weeding device is substantially horizontal, or more horizontal than vertical (i.e., at greater than 45° from a vertical orientation). Other approaches for using the orientation sensor 926 to selectively prevent or permit operation of the radiation source 920 are also considered.

With reference to FIG. 9B, another embodiment of the handheld weeding device 900 is illustrated. In this embodiment, the weeding device 900 includes a radio-frequency (RF) source 950. The RF source 950 can include one or more directional antenna disposed within the shaft 910 and/or otherwise coupled thereto for directing the radiation produced by the RF source 950 through the shaft 910 and toward the second end 914 thereof. The RF source 950 can be controlled similarly to the radiation source 920, for instance via the actuation device 930, the orientation sensor 926, and by an imaging device, such as the imaging device 924 described hereinabove. The RF source 950 can also be powered via the power connector 916, for instance via electrical power from a power cable 918. Other variants are also considered.

The methods for performing pest control among a plurality of plants described herein may be implemented in a high level procedural or object-oriented programming or scripting language, or a combination thereof, to communicate with or assist in the operation of a computer system, for example the computing device 710. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 712 of the computing device 710, to operate in a specific and predefined manner to perform the functions described herein, for example those described in the method 200.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure.

Various aspects of the methods and systems described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be obvious to those skilled in the art that changes and modifications may be made without departing from this invention in its broader aspects. The scope of the following claims should not be limited by the embodiments set forth in the examples, but should be given the broadest reasonable interpretation consistent with the description as a whole.

1. An autonomous unmanned ground vehicle (AUGV), comprising:
a chassis with a drive mechanism to displace the AUGV among a plurality of plants, the plurality of plants comprising at least one weed;
an image-capture device mounted to the chassis to obtain images of the plurality of plants;
a motorized arm extending between a proximal end mounted to the chassis and a distal free end, the free end of the arm being displaceable with respect to the chassis;
a microwave emitter mounted to the free end of the arm and displaceable therewith, the microwave emitter being operable to emit microwaves; and
a control system to operate at least the drive mechanism, the image-capture device, the motorized arm, and the microwave emitter, the control system comprising:
a processing unit; and
a non-transitory computer-readable memory having stored thereon instructions executable by the processing unit for causing the AUGV to perform at least:
independently navigating the chassis among the plurality of plants;
identifying the at least one weed in the images obtained by the image-capture device;
displacing the motorized arm to position the free end thereof in proximity to the at least one weed; and
emitting microwaves from the microwave emitter toward the at least one weed.

2. The AUGV of claim 1, further comprising a lens affixed to the microwave emitter to focus the microwaves into a microwave beam, wherein emitting microwaves from the microwave emitter toward the at least one weed comprises directing the microwave beam toward the at least one weed. The AUGV of claim 2, further comprising a waveguide affixed to the microwave emitter wherein

3. The AUGV of any one of claims 1 to 3, wherein the microewave beam toward the a t least one weed.
The AUGV of claim 4,

4. wherein the instructions cause the AUGV to identify the at least one weed by:
transmitting, over a data connection, at least some of the images to a remote server; and
receiving, over the data connection, an indication of the at least one weed in the images from the remote server.

5. The AUGV of claim 1, wherein the instructions cause the AUGV to identify at least one target area of the at least one weed, and to emit microwaves toward the at least one target area of the at least one weed.

6. The AUGV claim 1, wherein the image-capture device comprises at least one visible-light camera and at least one infrared camera.

7. The AUGV of one LiDAR device.

8. The AUGV of claim 1, wherein the instructions cause the AUGV to independently navigate the chassis among the plurality of plants by:
determining, via a global positioning system, a current location of the chassis;
identifying a location of interest within a predetermined area in which the plurality of plants are located;
determining a course from the current location of the chassis toward the location of interest; and
navigating the chassis to the location of interest.

9. A system for performing weeding among a plurality of plants comprising,
an AUGV according to claim 1; and
at least one server communicatively coupled to the AUGV and configured for receiving, over a data connection, at least some of the images from the AUGV and for transmitting, over the data connection an indication of the at least one weed to the AUGV.

10. A system for performing weeding amount a plurality of plants, comprising:
a plurality of AUGVS according to claim 1; the the
when AUGVs to autonomously synchronize the navigating.

11. A method for performing weeding among a plurality of plants, comprising:
obtaining images of at least some of the plurality of plants;
identifying at least one weed in the images of the at least some of the plurality of plants;
displacing a microwave source to position an emitter thereof in proximity to the at east one weed; and
emitting microwaves from the emitter toward the at least one weed.

12. The method of claim 11, wherein emitting microwaves comprises focusing the microwaves into a microwave beam and directing the microwave beam toward the at least one weed.

13. The method of claim 11
wherein identifying the at least one weed comprises:
transmitting, over a data connection, at least some of the images to a remote server; and
receiving, over the data connection, an indication of the at least one weed in the images from the remote server.

14. The method of claim 11, wherein identifying the at least one weed comprises identifying at least one target area of the at least one weed, and wherein emitting microwaves comprises emitting microwaves toward the at least one target area of the at least one weed.

15. The method of claim 11, wherein obtain the images of the at least some of the plurality of plants comprises obtaining the images using at least one of a visible-light camera and an infrared camera.

16. The method of claim 11, wherein obtaining the images of the at least some of the plurality of plants comprises obtaining the images using at least one LiDAR device.

17. The method of claim 11, wherein the emitting of the microwaves form the emitting end is performed responsive to an actuator mechanism associated with the microwave source being actuated.

18. The method of claim 11, wherein the images are obtained by an autonomous unmanned ground vehicle (AUGV), the method further comprising autonomously navigating the AUGV among the plurality pf plants.

19. The method of any one of claim 18, wherein autonomously navigating the vehicle comprises:
determining, via a global positioning system, a current location of the vehicle;
identifying a location of interest within a predetermined area in which the plurality of plants are located;
determining a course from the current location of the vehicle toward the location of interest; and
displacing the chassis autonomously to the location of interest.

20. The method of any one of claim 19, wherein autonomously navigating a vehicle among the plurality of plants comprises providing the vehicle with geospatial data indicative of the predetermined area, wherein the geospatial data includes a plurality of waypoints for navigating the vehicle, wherein the waypoints are produced by artificial intelligence.

21-34. (canceled)
